(19) United States
(12) Patent Application Publication
(10) Pub. No.: US 2019/0133749 A1
(43) Pub. Date: May 9, 2019

(54) ARTIFICIAL BLOOD VESSEL WITH STENT AND USE THEREOF IN CLINICAL OPERATION OF STANFORD TYPE A AORTIC DISSECTION

(71) Applicant: Jilin University, Changchun (CN)

(72) Inventor: Kexiang Liu, Changchun (CN)

(21) Appl. No.: 15/980,967

(22) Filed: May 16, 2018

(30) Foreign Application Priority Data
Nov. 8, 2017 (CN) 201711088778.2

(51) Int. Cl. A61F 2/07; A61F 2/89

(52) U.S. Cl. CPC A61F 2/07; A61F 2002/828; A61F 2002/075; A61F 2/89

(57) ABSTRACT

An artificial blood vessel with stent for use in clinical operation of Stanford types A aortic dissection, which can simplify treatment and reduce risk of surgery. The artificial blood vessel has a wall thickness of 0.30-0.35 mm and a length of 15-20 cm. The proximal portion of it refers to a portion with about 5-10 cm in length and contains just the polyester artificial blood vessel, the distal portion of it refers to a portion with about 8-10 cm in length and contains the polyester artificial blood vessel and a plural of “W” shaped titanium-nickel alloy stents with self-expanding function fixed inside the blood vessel, and the blood vessel and the stents are fixed together by stitching. The artificial blood vessel with stent has a diameter of 24-30 mm. When used in a surgery, different diameters are selected according to the internal diameters of aortic artery of different patients.

### CROSS-REFERENCE TO RELATED APPLICATION

This non-provisional application claims priority to and benefit of, under 35 U.S.C. § 119(a), Patent Application No. 201711088778.2, filed in P.R. China on Nov. 8, 2017, the entire content of which is hereby incorporated by reference.

### TECHNICAL FIELD

The present invention relates to an artificial blood vessel with stent and the use of it in clinical operation of Stanford type A aortic dissection.

### BACKGROUND

Aortic dissection refers to a phenomenon wherein the intima of aorta is torn by internal and external force, causing circulating blood enters between the intima layer and the middle layer of the aorta, causing separation of the two layers, as a result, an aortic dissection is formed. Aortic dissection is also known as aortic dissection aneurysm. The incidence of this disease is about 50 to 100 cases per 100,000 people per year, of which most of them involve a rapid onset. 65% to 75% of patients died of cardiovascular complications such as dissection rupture, cardiac tamponade, arrhythmia and the like within two weeks. The morbidity rate in male is higher than that in female, and the ratio of the former to the latter is 2-3:1. More than 80% of patients with aortic dissection have hypertension, and many patients have aortic cystic medial necrosis. Hypertension is not a factor causing the cystic medial necrosis but promotes its development, and the etiology behind it is still unknown.

There are two sets of classifications of the disease according to the sites of the laceration and the areas that the aortic dissection involves. That is, DeBakey classification, namely DeBakey I, II, and III types, and Stanford classification, namely Stanford A and Stanford B types. A dissection that involves the ascending aorta and the aortic arch is called the Stanford type A. Stanford type A dissection is in rapid onset with fierce progression, and about 1% of the patients die every hour within the first 24 hours of onset; if untreated, 50% of the patients die within 1 week, 70% of the patients die within two weeks, and 90% of the patients die within one year.

Since Stanford type A aortic dissection is critical, and there is a risk that aortic rupture might occur at any time, once diagnosed, timely and effective treatment must be performed. Therefore, how to choose a safe and effective method has been a hot research point in the field of cardiovascular surgeries and a challenge to cardiovascular surgeons. In 1983, Borst pioneered the treatment of Stanford type A aortic dissection with “elephant trunk implantation”. In recent years, with the advance of artificial blood vessel with stent, a new surgical method has been used frequently: ascending aorta replacement+total aortic arch replacement with artificial blood vessel+elephant trunk implantation. The use of this method has increased curing rate and reduced mortality rate. FIG. 1 is a schematic diagram of an artificial blood vessel with stent in the prior art. Some surgeons use pure artificial blood vessels as “soft elephant trunks”, and the others use artificial blood vessels with stents as “hard elephant trunks”. In both cases, the artificial blood vessels as “elephant trunks” are implanted into the descending aorta far beyond the left subclavian artery. Moreover, total aortic arch replacement with artificial blood vessel requires a three-branched artificial blood vessel, therefore several (at least five) vascular anastomotic sites have to be completed (as shown in FIG. 1), resulting in complicated surgical procedures, prolonged operation time, extracorporeal circulation time, systemic circulatory arrest time, and brain protection time, and many neurological and other complications. It is especially risky because it is difficult to control postoperative bleeding, as a result, serious consequences, including death of patients, can occur. A large number of research literatures have reported that the above method is used for the treatment of Stanford type A aortic dissection but since a three-branched artificial blood vessel is required for aortic arch replacement, the surgical procedure is complicated. Bleeding and complications in nervous system due to the great number of anastomotic sites tend to occur, and the procedure is very difficult to handle. Therefore, how to improve the surgical equipments and surgical methods in order to simplify the operation, makes it easy to handle has become an immanent issue to be considered for the treatment of Stanford type A aortic dissection.

### CONTENTS OF THE INVENTION

The object according to certain embodiments of the present invention is to solve the defects in the prior art and to provide a novel artificial blood vessel with stent. This invention can simplify the treatment of Stanford type A aortic dissection and reduce the operational risk.

In certain aspects, the present invention relates to an artificial blood vessel with stent, the blood vessel has a wall thickness of 0.30-0.35 millimeter (mm), a length of 15-20 centimeter (cm) and is a polyester artificial blood vessel; wherein the proximal portion thereof is a simple polyester artificial blood vessel with a length of about 5-10 cm, the distal portion thereof has a length of about 8-10 cm and contains a surface of polyester artificial blood vessel and has “W” shaped titanium-nickel alloy stents with self-expanding function inside, which are fixed by means of stitching; the artificial blood vessel with stent has a diameter of 24-30 mm; in a surgery, an artificial blood vessel with stent having a different diameter is selected according to the internal diameters of aortic artery of different patients.

In certain embodiments, the artificial blood vessel with stent has a specific diameter of 24 mm, 26 mm, 28 mm or 30 mm.

In certain embodiments, the distance between every two “W” shaped titanium-nickel alloy stents is of 5 mm.

In certain embodiments, the “W” shaped titanium-nickel alloy metal stent has a width of 7 mm.

In certain embodiments, when the blood vessel is not in use, i.e., not released, the “W” shaped titanium-nickel alloy stents are bound to a guide wire and an arc guide frame by threads; when used, the thread can be taken out through the guide wire to release the artificial blood vessel with stent with self-expanding function.

In certain aspects, the present invention relates to use of an artificial blood vessel with stent in clinical operation of Stanford type A aortic dissection.

Certain embodiments of the present invention provide an artificial blood vessel with stent, which is easy for handling, easy for use, and has beneficial effects. By using this new artificial blood vessel with stent, Stanford A-type aortic dissection is treated with internally-covering aortic arch replacement. Total aortic arch replacement can be achieved without the need to perform replacement of three branches of the aortic arch (FIG. 1). This can simplify the operation and make it easy to complete, therefore provides important clinical value.

### BRIEF DESCRIPTION OF THE DRAWINGS

FIG. 1 is a schematic diagram of an artificial blood vessel with stent in the prior art.

FIG. 2 is a schematic diagram of the artificial blood vessel with stent according to certain embodiments of the present invention.

FIG. 3 is a structural view of the artificial blood vessel with stent according to certain embodiments of the present invention, of which, “1” points to a simple polyester artificial blood vessel; “2” points to an artificial blood vessel with stent; “3” points to a stent;

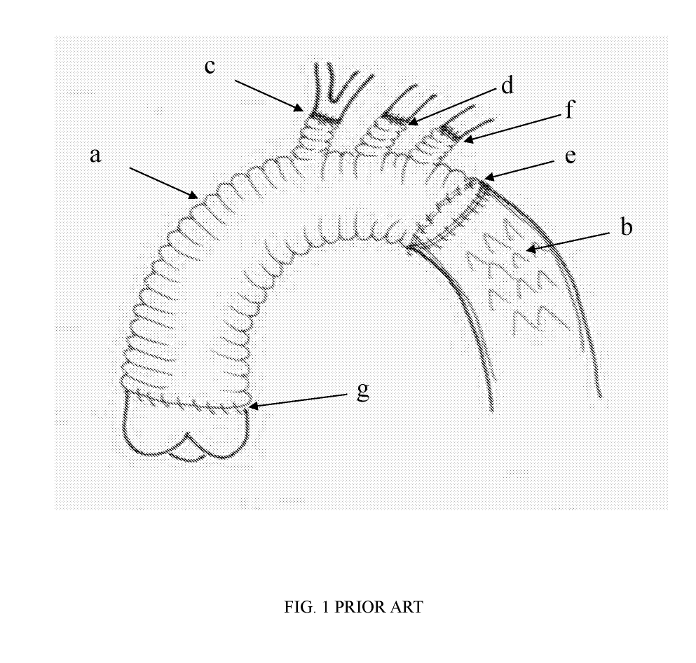

c, d, e, f and g: five anastomotic sites to be completed during the operation using the artificial blood vessel with stent existing in prior art;

a: an artificial blood vessel with three branches to be used to replace the ascending aorta and aortic arch;

b: an artificial blood vessel with stent to be implanted into the true lumen of the descending aorta;

c: an anastomotic site between an innominate artery and a branch of the artificial blood vessel;

d: an anastomotic site between the left common carotid artery and a branch of the artificial blood vessel;

e: an anastomotic site between the artificial blood vessel with stent and the artificial blood vessel with three branches, wherein the anastomotic site is located in the descending aortic arch at the distal end of the left subclavian artery; since the location is deep, operation for the anastomosis is difficult, and it is not easy to stop bleeding;

f: an anastomotic site between the left subclavian artery and a branch of the artificial blood vessel;

g: an anastomotic site between the artificial blood vessel with three branches and the aortic root.

D, E and G: three anastomotic sites to be completed during the operation using the artificial blood vessel with stent of the present invention;

D: an anastomotic site between the wedge-shaped end edge of the proximal artificial blood vessel of the stented blood vessel and the wall of descending aorta (continuously stitched with 5-0 prolene thread), after a portion of the artificial blood vessel which corresponds to the openings of the innominate artery, the left common carotid artery and the left subclavian artery is cut off;

E: an anastomotic site between the proximal broken end of the blood vessel at the arch and a second artificial blood vessel;

F: the second artificial blood vessel for replacing the ascending aorta; and

G: an anastomotic site between the second artificial blood vessel and the proximal end of ascending aorta.

### DETAILED DESCRIPTION OF THE INVENTION

The present invention will be described in detail below by way of specific examples.

An artificial blood vessel with stent according to certain embodiments of the present invention has a wall thickness of 0.30-0.35 mm, a length of 15-20 cm and is made of polyester.

Polyester is obtained by spinning and post-treating polyethylene glycol terephthalate (PET). PET is obtained through esterification or transesterification and condensation polymerization, using pure terephthalic acid (PTA) or Dimethyl Terephthalate (DMT) and ethylene glycol (EG) as raw materials.

The proximal portion of the artificial blood vessel refers to a portion with a length of about 5-10 cm and contains just the polyester artificial blood vessel. The aortic arch is internally covered with it in a surgery. This length is designed according to the length of the aortic arch. In order to make the proximal portion of the artificial blood vessel covers the aortic arch as much as possible, the length thereof should exceed the three branches of the aortic arch. However, since it is a waste if exceeding too much, this portion of artificial blood vessel is designed to have a length of about 5-10 cm.

The distal portion of the artificial blood vessel is an extended section of the proximal portion of the artificial blood vessel, and it contains the polyester artificial blood vessel and a plural of “W” shaped titanium-nickel alloy stents with self-expanding function fixed inside the blood vessel. During surgery, the distal portion is placed into the distal descending aorta of the left subclavian artery. The distal portion has a length of about 8-10 cm. This length is easy for handling and good therapeutic effects can be achieved. If the distal portion is too long, it is not conducive to be placed into the descending aorta, and too much intercostal artery openings would be covered, and thus complications such as bone marrow ischemia are prone to occur; if the distal portion is too short, it is not conducive to cover the intimal tear of the dissection, supporting effect is also small, making against expansion of the true lumen of the dissection.

The “W” shaped titanium-nickel alloy stents are fixed inside the distal portion of the artificial blood vessel by stitching.

A: the distance between every two “W” shaped titanium-nickel alloy stents, 5 mm; and the distance between two groups of metal stents is of 5 mm. The portion of the artificial blood vessel without stents can make such artificial blood vessel with stent to have better compliance and to be more suitable for walking course, curvature and lumen size of aorta in different patients.

B: the width of the “W” shaped titanium-nickel alloy metal stent is 7 mm. The multiple layers of the “W” titanium-nickel alloy metal stent with self-expanding function can increase the supporting effect of the artificial blood vessel, increase the true lumen of aorta, reduce the false lumen, and promote the healing of the dissection.

C: the diameter of the artificial blood vessel with stent is 24-30 mm, in particular it is 24 mm, 26 mm, 28 mm or 30 mm. A vast majority of patients has an aortic internal diameter within this range, and an artificial blood vessel with stent having a different diameter is selected according to the internal diameter of aorta of different patients. If the diameter is too small, the blood vessel cannot generate supporting effect, the artificial blood vessel is not closely attached to the intima of aorta, and internal fistula easily occurs; if the diameter is too large, excessive expansion of the aorta may be caused, and the intima might be torn, resulting in new dissection or aortic rupture.

After the circulation of the internally-covering aortic arch replacement surgery in a Stanford A-type aortic dissection is stopped, the ascending aorta is transected at the proximal end of the innominate artery, the new artificial blood vessel with stent is placed into the descending aorta from the true lumen of the aortic arch, via the transected ascending aorta. The proximal end of the stents is aligned with the lower edge of the opening of the left subclavian artery to release the new artificial blood vessel with stent. After the artificial blood vessel with stent, as an elephant trunk, is released, the distal end of the stent expands spontaneously; it then expands the proximal end of the artificial blood vessel. A part of the artificial blood vessel that corresponds to the openings of the innominate artery, the left common carotid artery, and the left subclavian artery are then being cut off with a wedge shape, fully exposing the openings of the left subclavian artery, the left common carotid artery, and the innominate artery; the wedge-shaped edge at the proximal end of the stented artificial blood vessel is anastomosed with the inner wall of the aortic arch by continuously stitching with 5-0 prolene thread. In this case, except for the portion at the openings of the three-branched vessels, the inner wall of the aortic arch is almost completely covered by the artificial vessel, which is equivalent to an aortic arch replacement with an island anastomosis. After that, the ascending aorta is replaced with another artificial blood vessel.

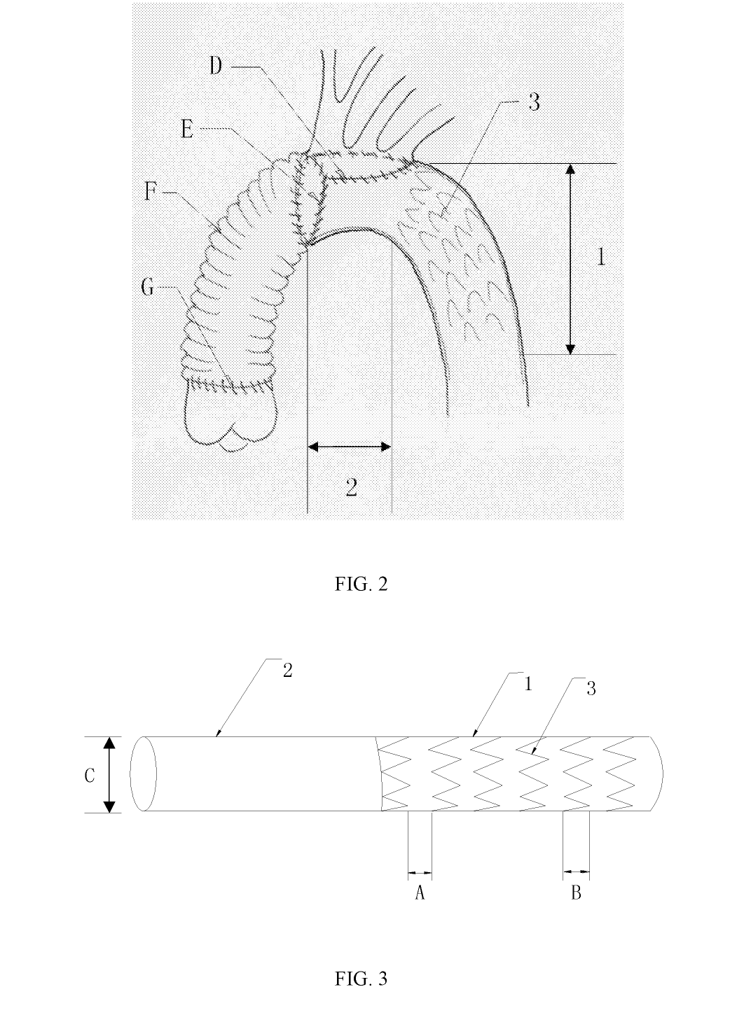

In the clinical treatment of Stanford type A aortic dissection, the use of internally-covering aortic arch replacement surgery can significantly simplify the traditional total aortic arch replacement surgery, without performing anastomosis of three branched blood vessels of the aortic arch, so that five anastomosis are reduced to two anastomosis (E and G in FIG. 2), and an anastomotic site that is originally located at the distal end of the left subclavian artery with a deep position which is difficult to operate and not easy to stop bleeding is moved to the proximal end of the innominate artery, and thus the operation is obviously simplified, and it is easier to stop the bleeding, obviously reducing the obstinate bleeding problems which might be caused by total aortic arch replacement surgery, reducing the extracorporeal circulation time, reducing the operation time, improving the success rate of surgery, reducing blood use while reducing the complications caused by massive blood transfusion. This enables such large and complex cardiovascular surgery becoming a common surgery, facilitating the widespread popularization of the surgery.

The present invention is further illustrated by the following examples. It should be understood, however, that the examples are for illustration purposes only and are not intended to limit the scope and spirit of the invention.

### Embodiment 1

A patient, male, 48 years old, with emergency admission due to intermittent distending pain in chest and abdomen for 6 hours. Clinical diagnosis: acute aortic dissection (Stanford A type), hypertension 3 (very high risk group). After preoperative preparation, with general anesthesia and extracorporeal circulation, the ascending aortic replacement with the artificial blood vessel with stent+internally covering aortic arch replacement+elephant trunk implantation with the artificial blood vessel with stent were employed.

After intraoperative general anesthesia, the sternum was incised in the middle, and the right axillary artery, femoral artery and superior and inferior vena cava were catheterized. Accordingly, an extracorporeal circulation was established. The aortic arch, innominate artery, left common carotid artery and left subclavian artery were separated, and bagged for ready to use. The left ventricular drainage was conducted through the right pulmonary vein. The temperature was lowered, the ascending aorta was blocked and incised, a myocardial protective solution was perfused through the openings of the left and right coronary arteries, cardiac arrest occurred, ice debris was placed on the surface of the heart, the proximal end was trimmed, and the proximal ascending aorta wall was reinforced by means of sandwiches procedure. When the rectal temperature dropped to 28° C., the circulation was stopped, the ascending aorta was transected at the proximal end of the innominate artery and bilateral selectively cerebral perfusion was performed by means of cannulation through axillary artery—right common carotid artery and left common carotid artery. It was found that: an annular, irregular intimal tear with a length of about 5 cm was found at the curvature side in the junction of the ascending aorta and the aortic arch, and the dissection lesion was involved in the aortic arch at the distal end, while the innominate artery, left common carotid artery and left subclavian artery were generally not too bad, and the dissection lesion was peeled to the distal reaching the descending aorta. The artificial blood vessel with stent (28 mm in diameter, 10 cm in length for the distal stented portion, and 10 cm for the proximal artificial blood vessel) was inserted from the true lumen of the dissection through the transected ascending aorta, and the proximal end aligned with the underside of the opening of the left subclavian artery to release the stented blood vessel as an elephant trunk. A part of the artificial blood vessel which corresponds to the openings of the innominate artery, the left common carotid artery and the left subclavian artery is cut off, fully exposing the openings of left subclavian artery, left common carotid artery, innominate artery. The excess artificial blood vessel beyond the broken end of the aorta was cut off. The wedge-shaped edge at the proximal end of the stented artificial blood vessel is anastomosed with the wall of descending aortic arch by means of continuously stitching with 5-0 prolene thread. An 1×6 cm artificial blood vessel patch was placed inside the proximal portion of the innominate artery, and an artificial blood vessel patch with a width of 1 cm was placed outside the proximal portion of the aorta in the arch, and the proximal broken end of blood vessel in the arch was reinforced by means of continuously stitching with 4-0 prolene thread. In this case, except for the portion at the openings of three branched vessels, the wall of the aortic arch is almost completely covered by the artificial vessel, which corresponds to a reestablished aortic arch replacement for the island three branched blood vessels. The ascending aorta was then replaced with a 26 mm artificial blood vessel with a single-branch (this artificial vessel is unrelated to the new artificial blood vessel with stent of the present invention). Intraoperative operation was smooth, postoperative recovery was good, and the patient was cured and discharged on the 18^{th} day after the operation.

### Embodiment 2

A patient, male, 68 years old, with emergency admission due to severe pain in thorax and abdomen for 3 days and aggravation for 1 day. Clinical diagnosis: acute aortic dissection (Stanford A type). After preoperative preparation, under general anesthesia and extracorporeal circulation, the ascending aortic replacement with the artificial blood vessel with stent+internally covering aortic arch replacement+elephant trunk implantation with the artificial blood vessel with stent were employed.

After intraoperative general anesthesia, the sternum was incised in the middle, and the right axillary artery, femoral artery and superior and inferior vena cava were subjected to catheterization. Accordingly, an extracorporeal circulation was established. The aortic arch, innominate artery, left common carotid artery and left subclavian artery were separated, and bagged for ready to use. The left ventricular drainage was conducted through the right pulmonary vein. The temperature was lowered, the ascending aorta was blocked and incised, a myocardial protective solution was perfused through the openings of the left and right coronary arteries, cardiac arrest occurred, and ice debris was placed on the surface of the heart. It was found that: an irregular intimal tear with a length of about 6 cm was at the posterior ascending aorta wall, and the dissection lesion was involved in the aortic arch at the distal end. The proximal end was trimmed, and the proximal ascending aorta wall was reinforced by means of sandwiches procedure. When the rectal temperature dropped to 28° C., the circulation was stopped, the ascending aorta was transected at the proximal end of the innominate artery and bilateral selectively cerebral perfusion was performed by means of cannulation through axillary artery—right common carotid artery and left common carotid artery. It was found that: the dissection lesion was involved in the aortic arch at the distal end, while the innominate artery, left common carotid artery and left subclavian artery were generally not bad, and the dissection lesion was peeled to the distal reaching the descending aorta. The artificial blood vessel with stent (26 mm in diameter, 10 cm in length for the distal stented portion, and 10 cm for the proximal artificial blood vessel) was inserted from the true lumen of the dissection through the transected ascending aorta, and the proximal end aligned with the underside of the opening of the left subclavian artery to release the stented blood vessel as an elephant trunk. A part of the artificial blood vessel which corresponds to the openings of the innominate artery, the left common carotid artery and the left subclavian artery is cut off, fully exposing the openings of left subclavian artery, left common carotid artery, innominate artery. The wedge-shaped end edge of the proximal artificial blood vessel of the stented blood vessel is anastomosed with the wall of aortic arch by means of continuously stitching with 5-0 prolene thread. An 1×5 cm artificial blood vessel patch was placed inside the proximal portion of the innominate artery, an artificial blood vessel patch with a width of 1 cm was placed outside the proximal portion of the aorta in the arch, and the proximal broken end of blood vessel in the arch was reinforced by means of continuously stitching with 4-0 prolene thread. In this case, except for the portion at the openings of three branch vessels, the wall of the aortic arch is almost completely covered by the artificial vessel, which corresponds to a reestablished aortic arch replacement for the island three branch blood vessels. The ascending aorta was then replaced with a 26 mm artificial blood vessel with a single-branch (this artificial vessel was unrelated to the novel artificial blood vessel with stent of the present invention). Intraoperative operation was smooth, postoperative recovery was good, and the patient was cured and discharged on the 14^{th} day after the operation.

1. An artificial blood vessel with stent, having a length of about 15-20 cm, a diameter of 24-30 mm, and a wall thickness of 0.30-0.35 mm, wherein the artificial blood vessel comprises:
a proximal artificial blood vessel portion, having a length of about 5-10 cm and made of polyester; and
a distal artificial blood vessel portion, having a length of longer than 8 cm and less than 10 cm and made of a polyester surface and a plurality of “W” shaped titanium-nickel alloy stents with self-expanding function fixed inside the polyester surface,
wherein the polyester surface and the stents are stitched together, and the diameter of the artificial blood vessel with stent is selected based on internal diameters of aortic artery of different patients.

2. The artificial blood vessel with stent according to claim 1, wherein the artificial blood vessel has a diameter of about 24 mm, 26 mm, 28 mm or 30 mm.

3. The artificial blood vessel with stent according to claim 1, wherein a distance between each of the “W” shaped titanium-nickel alloy stents is 5 mm.

4. The artificial blood vessel with stent according to claim 1, wherein the “W” shaped titanium-nickel alloy metal stent has a width of 7 mm.

5. A use of the artificial blood vessel with stent of claim 1 in clinical operation of Stanford type A aortic dissection.
